SEQUENCE LISTING (2 sequences)

SEQ ID NO: 1 - DNA, 18 bp, Artificial Sequence
  primer
gaccacgcccaacaagct

SEQ ID NO: 2 - DNA, 29 bp, Artificial Sequence
  primer
agcatgaattgttaacctcattgtataga

(19) United States
(12) Patent Application Publication
(10) Pub. No.: US 2017/0198359 A1
(43) Pub. Date: Jul. 13, 2017

(54) METHODS AND COMPOSITIONS FOR TREATMENT OR DIAGNOSIS OF CANCERS RELATED TO GABRA3

(71) Applicant: The Wistar Institute of Anatomy and Biology, Philadelphia, PA (US)

(72) Inventors: Qihong Huang, Ardmore, PA (US); Kazuko Nishikura, Haddonfield, NJ (US); Kiranmai Gumireddy, Newtown Square, PA (US)

(21) Appl. No.: 15/397,506

(22) Filed: Jan. 3, 2017

Related U.S. Application Data
(60) Provisional application No. 62/276,109, filed on Jan. 7, 2016.

(51) Int. Cl. C12Q 1/68; A61K 31/365; A61K 31/5517; G01N 33/574

(52) U.S. Cl. CPC C12Q 1/6886; G01N 33/57415; A61K 31/365; A61K 31/5517; G01N 2333/70571; C12Q 2600/158; C12Q 2600/118

(57) ABSTRACT

Compositions and methods for diagnosing and treating breast cancer, including metastatic breast cancer, are provided. In one aspect, a diagnostic composition comprising a reagent which is capable of specifically complexing with, or identifying, GABRA3 is provided. In another aspect, a method of detecting breast cancer in a subject comprising measuring the level of GABRA3 in a biological sample from the subject is provided. In yet another aspect, a method of treating breast cancer is provided, the method comprising: measuring the level of GABRA3 in a biological sample from a subject and treating the subject with a reagent that inhibits the action of GABA when GABRA3 is detected in the sample or when there is an increase in the level of GABRA3 in the sample as compared to a control sample from a healthy subject.

### STATEMENT OF GOVERNMENT INTEREST

This invention was made with government support under Grant No. CA010815 awarded by the National Institutes of Health. The government has certain rights in the invention.

### BACKGROUND

While metastasis remains the major cause of death from cancer, the critical molecular controls underlying tumor metastasis are only poorly understood. Identification of novel key regulators of metastasis and designing new ways to target and inhibit those proteins are likely to have profound benefits to the survival of cancer-affected individuals.

Chloride channels are responsible for the active transportation of chloride across the plasma membrane (1). Chloride functions in an electrochemical equilibrium, and serves as an important signaling molecule in most cells (1). Dysfunction of chloride transport is associated with a number of human diseases including cystic fibrosis. However, the functions of chloride channels in cancer development have not been studied extensively. Gamma-aminobutyric acid (GABA) is an inhibitory neurotransmitter (2). GABA exerts its function through two types of GABA receptors: ionotropic receptor including GABA_{A} receptor and GABA_{C} receptor; and metabotropic GABA_{B} receptor (3). GABA_{A} receptor is a pentamer comprised of various subunits and functions as a chloride channel (3). It was reported that expression of the GABA_{A} receptor, the GABA transporter, and the GABA transaminase is up-regulated in brain metastases of breast cancer (4). These metastatic cells display a GABAergic phenotype similar to that of neuronal cells and possibly use GABA for their proliferation (4). However, whether and how the GABA_{A} receptor and its signaling pathways function in cancer development and metastasis are largely unknown.

The enzyme ADAR was originally detected as a dsRNA unwinding activity in Xenopus eggs and embryos (5, 6) and was later found as a dsRNA-specific adenosine deaminase (7, 8). These discoveries opened up the previously unrecognized field of A-to-I RNA editing (9-17). ADARs specifically target dsRNAs and deaminate adenosine residues to inosine via a hydrolytic deamination reaction (A-to-I RNA editing). The edited inosine residue in RNA is detected as an A-to-G change in the cDNA sequence, and the translation machinery also reads inosine as guanosine, leading to alterations of codons. Interestingly, the coding region of chloride channel GABA_{A} receptor 3 (GABRA3), one of the subunits of GABA_{A} receptor, undergoes A-to-I editing which results in one amino acid change in GABRA3 protein (18). However, the functions of A-to-I edited GABRA3 in cancer development have not been studied.

GABRA3 is normally expressed in neuronal, not breast epithelial, cells. Notably, there is precedence for neuronal proteins becoming aberrantly expressed in cancer and contributing to metastasis. Importantly, there is also precedence for therapeutic targeting of these aberrantly expressed proteins for cancer treatment. Specifically, the glutamate receptor GRM1, predominantly expressed in the brain, is aberrantly overexpressed in melanoma (30). Moreover, suppression of GRM1 and the glutamate neurotransmission pathway decreases melanoma progression (31). Riluzole, an FDA-approved drug that inhibits the release of glutamate and is used for the treatment of amyotrophic lateral sclerosis, has shown significant anti-tumor activity for melanoma treatment in clinical trials (32, 33). These results indicate that a gene that is specifically expressed in one tissue but aberrantly expressed in tumors of another tissue and plays important roles in tumor development, can serve as an important therapeutic target.

While GABRA3 expression has been associated with other cancers, namely hepatocellular carcinoma (Liu et al, Gamma-aminobutyric acid promotes human hepatocellular carcinoma growth through overexpressed gamma-aminobutyric acid A receptor α3 subunit, World J Gastroenterol 2008 Dec. 21; 14(47): 7175-7182), and lung cancer (Liu et al, Gammaaminobutyric Acid A Receptor Alpha 3 Subunit is Overexpressed in Lung Cancer, Pathol. Oncol. Res. (2009) 15:351-358 (Liu 2009)), it has been shown that GABRA3 is not expressed in breast cancer tissue (Liu 2009).

What is needed are reagents for diagnosing and therapeutic targets for treating metastatic breast cancer.

### SUMMARY OF THE INVENTION

In one aspect, a diagnostic composition is provided which includes a reagent which is capable of specifically complexing with, or identifying, GABRA3. In one embodiment, the reagent is covalently or non-covalently joined with a detectable label or with a substrate. In one embodiment, the reagent comprises a polynucleotide or oligonucleotide sequence, a protein or peptide, or antibody or fragment thereof.

In another aspect, a method of detecting breast cancer in a subject is provided. The method includes measuring the level of GABRA3 in a biological sample from the subject, wherein the presence of GABRA3 in the sample is indicative of breast cancer.

In yet another aspect, a method of detecting breast cancer in a subject is provided. The method includes measuring the level of GABRA3 in a biological sample from the subject, wherein an increase in the level of GABRA3 in the sample as compared to a control, is indicative of breast cancer.

In another aspect, a method of detecting the risk of breast cancer metastasis in a subject is provided. The method includes measuring the level of GABRA3 in a biological sample from the subject, wherein an increase in the level of GABRA3 in the sample as compared to a control, is indicative of an increased risk of metastasis.

In yet another aspect, a method of detecting the risk of breast cancer metastasis in a subject is provided. The method includes measuring the level of A-to-I RNA edited GABRA3 in a biological sample from the subject, wherein an decrease in the level of A-to-I RNA edited GABRA3 in the sample as compared to a control, is indicative of an increased risk of metastasis.

In another aspect, a method of treating breast cancer is provided. In one embodiment, the method includes inhibiting the action of GABA. In another embodiment, the method includes decreasing GABRA3 levels in a subject. In yet another embodiment, the method includes administering a GABAA receptor antagonist.

In yet another aspect, a method of treating breast cancer is provided. The method includes increasing the expression of A-to-I edited GABRA3.

In another aspect, a method of treating breast cancer is provided. The method includes measuring the level of GABRA3 in a biological sample from a subject and treating the subject with a reagent that inhibits the action of GABA when GABRA3 is detected in the sample or when there is an increase in the level of GABRA3 in the sample as compared to a control sample from a healthy subject. In one embodiment, the reagent that inhibits the action of GABA is a GABAA receptor antagonist.

In another aspect, a method of reducing migration and/or invasion of breast cancer cells is provided. The method includes inhibiting the action of GABA or decreasing the level of expression or activity of GABRA3. In one embodiment, GABRA3 levels are decreased or the action of GABA is inhibited using a GABAA receptor antagonist. In another embodiment, GABRA3 levels are decreased or the action of GABA is inhibited by increasing the expression of A-to-I edited GABRA3.

In yet another aspect, the use of a composition comprising a reagent that decreases GABRA3 levels, for the manufacture of a medicament for use in treating breast cancer is provided.

In another aspect, a method of treating breast cancer is provided. In one embodiment, the method includes administering a composition that decreases the level of GABRA3 and a composition that increases the expression of A-to-I edited GABRA3.

Other aspects and advantages of these compositions and methods are described further in the following detailed description of the preferred embodiments thereof.

### BRIEF DESCRIPTION OF THE DRAWINGS

FIGS. 1A-D show that GABRA3 is highly expressed in human breast cancer cells and tissues but not in normal human breast tissues and cells. High GABRA3 expression is inversely correlated with survival in breast cancer. (A) GABRA3 is expressed only in the brain of normal human adult tissues. (B) GABRA3 is expressed in human breast cancer cell lines but not in normal human breast epithelial cells. (C) GABRA3 is highly expressed in metastatic breast cancer tissues than primary cancer tissues in paired clinical breast cancer samples. (D) High GABRA3 expression is significantly associated with poor survival in breast cancer (cox regression p=0.003, Hazard ratio=2.85).

FIGS. 2A-C show that GABRA3 promotes tumor cell migration, invasion and metastasis in breast cancer. (A-B) Human breast cancer MCF7 cells stably expressing GABRA3 were subjected to migration (A) and invasion (B) assays. The expression of GABRA3 in these cells leads to significant increase of cell migration and invasion. (C) Transplantation of human breast cancer MCF7 cells stably expressing GABRA3 in mouse mammary fat pads leads to lung metastasis whereas MCF7 cells expressing a vector control did not.

FIGS. 3A-C show that suppression of GABRA3 expression inhibits tumor cell migration, invasion, and metastasis in breast cancer. (A-B) Human breast cancer MDA-MB-436 cells stably expressing GABRA3 shRNAs were subjected to migration (A) and invasion (B) assays. The suppression of GABRA3 in these cells leads to significant decrease of cell migration and invasion. (C) Transplantation of human breast cancer MDA-MB-436 cells stably expressing GABRA3 shRNA in mice leads to the decrease of lung metastasis when compared with MDA-MB-436 cells stably expressing a control vector.

FIGS. 4A-D show that A-to-I RNA edited GABRA3 is expressed in non-invasive human breast cancer cells. (A) Sequencing of GABRA3 expressed in invasive human breast cancer MDA-MB-436 cells indicates GABRA3 was not RNA-edited. Arrow indicates the nucleotide of adenosine of the wildtype GABRA3. (B) Sequencing of GABRA3 expressed in non-invasive human breast cancer MCF7 cells indicates GABRA3 was RNA-edited. Arrow indicates the A-to-I edited nucleotide of GABRA3. (C) Percentage of A-to-I edited GABRA3 expressed in human breast cancer cell lines were determined by sequencing. A-to-I RNA edited GABRA3 is not expressed in invasive human breast cancer cells. (D) RNA-editing enzyme ADAR1 is expressed in human breast cancer cells and normal human breast epithelial cells.

FIGS. 5A-C show that A-to-I RNA-edited GABRA3 suppresses tumor cell migration, invasion, and metastasis in breast cancer. (A-B) Human breast cancer MCF7 cells stably expressing a control vector, or unedited GABRA3, or unedited GABRA3 and RNA-edited GABRA3, were subjected to migration (A) and invasion (B) assays. The expression of unedited GABRA3 in these cells leads to significant increase of cell migration (A) and invasion (B). The expression of RNA-edited GABRA3 reverses the phenotypes of wildtype GABRA3 (A-B). (C) Transplantation of human breast cancer MDA-MB-436 cells stably expressing RNA-edited GABRA3 in mice leads to the decrease of lung metastasis when compared with MDA-MB-436 cells stably expressing a control vector.

FIGS. 6A-D show that A-to-I RNA edited GABRA3 reduces GABRA3 expression on cell surface and suppresses AKT activation. (A) Representative flow cytometry histogram overlay of GABRA3 surface expression in MDA-MB-436. Human breast cancer MDA-MB-436 cells expressing RNA-edited GABRA3 (medium grey), or a control vector (light grey), were subjected to FACS analysis using a GABRA3 antibody or control IgG. The expression of RNA-edited GABRA3 decreases GABRA3 expression on cell surface. (B) Representative flow cytometry histogram overlay of GABRA3 surface expression in MCF7 cells. Human breast cancer MCF7 cells stably expressing a control vector (dark grey), or unedited GABRA3 (light grey), or unedited GABRA3 and RNA-edited GABRA3 (medium grey), were subjected to FACS analysis using a GABRA3 antibody or control IgG. The expression of unedited GABRA3 in these cells leads to significant increase of GABRA3 expression on cell surface. The expression of RNA-edited GABRA3 reverses the phenotypes of wildtype GABRA3. (C) Phosphorylated and total AKT in human breast cancer MDA-MB-436 cells expressing RNA-edited GABRA3, or a control vector, were determined by immunoblotting. The expression of RNA-edited GABRA3 inhibits AKT activation without affecting total AKT. (D) Phosphorylated and total AKT in human breast cancer MCF7 cells stably expressing a control vector, or unedited GABRA3, or unedited GABRA3 and RNA-edited GABRA3, were determined by immunoblotting. The expression of unedited GABRA3 in these cells leads to the activation of AKT. The expression of RNA-edited GABRA3 reverses the phenotypes of unedited GABRA3.

FIG. 7 shows the list of genes in which expression is significantly associated with survival in breast cancer samples.

FIG. 8 shows GABRA3 expression in MCF7 cells expressing GABRA3 or a control vector.

FIG. 9 shows GABRA3 expression in MDA-MB-436 cells expressing GABRA3 shRNA1, or GABRA3 shRNA2, or a control shRNA.

FIG. 10 shows MDA-MB-436 cells expressing GABRA3 shRNA1, or GABRA3 shRNA2, or a control shRNA, subjected to MTT assay. Knockdown of GABRA3 did not affect cell proliferation in MDA-MB-436 cells.

FIGS. 11A-B show MCF7 cells stably expressing GABRA3 treated with a pan-AKT inhibitor MK-2206 and cells were subjected to migration (A) and invasion (B) assay. MK-2206 suppressed cell migration and invasion.

FIG. 12 shows the results of MDA-MB-436 cells treated with GABRA3 inhibitors picrotoxin or flumazenil at various concentrations. Cells were subjected to Boyden chamber assays. The data show that GABRA3 inhibitors picrotoxin and flumazenil suppress cell migration and invasion.

### DETAILED DESCRIPTION OF THE INVENTION

It is demonstrated herein that high expression of GABRA3 is significantly inversely correlated with breast cancer survival. Further, it is shown that overexpression of GABRA3 promotes breast cancer cell migration, invasion and metastasis. Conversely, that knockdown of GABRA3 expression suppresses cell invasion and metastasis, but has no effect on cell proliferation. Importantly, it is demonstrated that GABRA3 is highly expressed in breast cancer cell lines and tissues but not in normal breast epithelial cells or normal breast tissue. Mechnistically, GABRA3 activates AKT pathway to promote cell migration and invasion. It is also shown that A-to-I editing of GABRA3 occurs only in non-invasive breast cancer cells. RNA-edited GABRA3 suppresses the functions of wildtype GABRA3 in cell invasion and metastasis.

### I. GENERAL

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The terms “a” or “an” refers to one or more, for example, “a GABAA antagonist” is understood to represent one or more GABAA antagonists. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is a female.

“Control” or “Control subject” as used herein refers to both an individual with normal breast tissue (i.e., normal individuals) or the pooled biological samples (e.g., tissue biopsy) from multiple normal individuals or numerical or graphical averages of the expression levels of the selected biomarkers obtained from large groups of normal individuals. In one embodiment, such control individuals are females. Such controls are the types that are commonly used in similar diagnostic assays for other biomarkers. Selection of the particular class of controls depends upon the use to which the diagnostic methods and compositions are to be put by the physician. As used herein, the term “predetermined control” refers to a numerical level, average, mean or average range of the expression of a biomarker in a defined population. The predetermined control level is preferably provided by using the same assay technique as is used for measurement of the subject's biomarker levels, to avoid any error in standardization.

In another embodiment, the control may be an individual (or population of individuals) who have or who have had, breast cancer. The control can refer to a numerical average, mean or average range of the expression of one or more biomarkers, in a defined population, rather than a single subject.

As used herein, the term “any intervening amount”, when referring to a range includes any number included within the range of values, including the endpoints.

“Sample” as used herein means any biological fluid or tissue that contains, or may contain GABRA3 as it relates to breast cancer. The most suitable samples for use in the methods and with the compositions are blood samples, including serum, plasma, whole blood, and peripheral blood; tissue samples, including biopsy samples; and any samples which contain breast cancer cells. It is also anticipated that biological samples which contain metastatic breast cancer cells (e.g., liver, lung, or brain cells or tissue) may be used. Further it is anticipated that other biological fluids, such as saliva or urine, and vaginal or cervical secretions may be used similarly. Such samples may further be diluted with saline, buffer or a physiologically acceptable diluent. Alternatively, such samples are concentrated by conventional means.

The term “microarray” refers to an ordered arrangement of hybridizable array elements, e.g., primers, probes, ligands, on a substrate.

The term “ligand” refers to a molecule that binds to a protein or peptide, and includes antibodies and fragments thereof.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “antibody” or “antibodies” as used herein refers to all types of immunoglobulins, including IgG, IgM, IgA, IgD, and IgE, including antibody fragments. The antibody can be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989). The antibodies can be recombinant monoclonal antibodies produced according to known methods, see, e.g., U.S. Pat. Nos. 4,474,893 or 4,816,567, which are incorporated herein by reference. The antibodies can also be chemically constructed according to known methods, e.g., U.S. Pat. No. 4,676,980 which is incorporated herein by reference. See also, U.S. Pat. No. 8,613,922, which is incorporated herein by reference.

Antibody fragments include, for example, Fab, Fab′, F(ab′)₂, and Fv fragments; domain antibodies, bifunctional, diabodies; vaccibodies, linear antibodies; single-chain antibody molecules (scFV); and multispecific antibodies formed from antibody fragments. Such fragments can be produced by known techniques.

Antibodies utilized herein may be altered or mutated for compatibility with species other than the species in which the antibody was produced. For example, antibodies may be humanized or camelized. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Methods for humanizing non-human antibodies are well known in the art. See, e.g., the method of Winter and co-workers (Jones et al., Nature 321:522 (1986); Riechmann et al., Nature 332:323 (1988); Verhoeyen et al., Science 239:1534 (1988)), each of which is incorporated herein by reference.

The term “reagent” or “ligand” refers to a molecule that binds, complexes, hybridizes or interacts with or to GABRA3 or a fragment thereof, or to the nucleic acid sequence encoding it or from which it is transcribed so as to identify the expression of GABRA3 in a cancer cell.

As used herein, “labels” or “reporter molecules” are chemical or biochemical moieties useful for labeling a nucleic acid (including a single nucleotide), polynucleotide, oligonucleotide, or protein ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand.

Gamma-aminobutyric acid (GABA) is the major inhibitory neurotransmitter in the mammalian brain where it acts at GABA-A receptors, which are ligand-gated chloride channels. Chloride conductance of these channels can be modulated by agents such as benzodiazepines that bind to the GABA-A receptor (also called herein, interchangeably, GABAA receptor and GABA_{A} receptor). At least 16 distinct subunits of GABA-A receptors have been identified, including the alpha 3 subunit, GABRA3. GABRA3 is a subunit of the GABAA receptors that may associate with other GABA_{A} receptor subunits to form a functional chloride channel that mediates the inhibitory synaptic transmission in the mature central nervous system (CNS). In the case of the GABA_{A} receptor, there are 16 related subunits (α1-6, β1-3, γ1-3, δ, ε, θ, π) that comprise the “classical” GABA_{A} receptor plus an additional three subunits (ρ1-3) that form the so-called GABA_{C} receptor. The GABA recognition site occurs at the interface of the α and β subunits and when a γ2 subunit is adjacent to either an α1, α2, α3 or α5 subunit, a benzodiazepine recognition site is formed. As used herein, GABRA3 refers to the α3 subunit of the GABAa receptor, including functional fragments thereof. See, Atack, J R, Development of Subtype-Selective GABAA Receptor Compounds for the Treatment of Anxiety, Sleep Disorders and Epilepsy, J. M. Monti et al. (eds.), GABA and Sleep, DOI 10.1007/978-3-0346-0226-6_2, # Springer Basel AG 2010, which is incorporated herein by reference.

### II. DIAGNOSTIC COMPOSITIONS

A variety of compositions and methods can be employed for the detection, diagnosis, monitoring, and prognosis of breast cancer, or the status of breast cancer, and for the identification of subjects with an increased risk of breast cancer metastasis. In one aspect, a diagnostic composition useful in diagnosing and/or treating breast cancer is provided. In one embodiment, the composition includes a ligand which is capable of specifically complexing with, or identifying, GABRA3, or the mRNA encoding the same, including a fragment or portion thereof.

There are a variety of assay formats known to the skilled artisan for using a binding agent to detect a target molecule in a sample. Any ligand which is capable of specifically complexing with, or identifying, GABRA3, or the mRNA encoding the same, including a fragment or portion thereof, which is useful in one or more of the various assay methods, is contemplated herein. In one embodiment, the ligand is a polynucleotide or oligonucleotide sequence, which sequence binds to, complexes with or identifies GABRA3 or the mRNA encoding the same, or a fragment thereof. In another embodiment, the ligand is a protein or peptide, which protein or peptide binds to, complexes with or identifies GABRA3 or the mRNA encoding the same or a portion or fragment thereof. In another embodiment, the ligand is an antibody or fragment thereof which binds to, complexes with or identifies GABRA3 or the mRNA encoding the same or a portion or fragment thereof.

As used herein, the term “antibody” refers to an intact immunoglobulin having two light and two heavy chains or any fragments thereof. Thus a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc. A recombinant molecule bearing the binding portion of an anti-GABRA3 antibody, e.g., carrying one or more variable chain CDR sequences that bind GABRA3, may also be used in a diagnostic assay. As used herein, the term “antibody” may also refer, where appropriate, to a mixture of different antibodies or antibody fragments that bind to GABRA3. Such different antibodies may bind to different biomarkers or different portions of GABRA3 protein than the other antibodies in the mixture.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

In yet another embodiment, the reagent is a primer set or primer-probe set capable of identifying and/or amplifying GABRA3 or a portion thereof. An example of a primer set capable of identifying and/or amplifying GABRA3 or a portion thereof is described in Example 1E. Such primers include GABRA3 Forward-5′-GACCACGCCCAACAAGCT-3′ (SEQ ID NO: 1) and Reverse-5″-AGCATGAATTGTTAACCTCATTGTATAGA-3′ (SEQ ID NO: 2). Other suitable primers can be designed by the person of skill in the art and/or obtained commercially.

In one embodiment, the reagent forms a complex with GABRA3. In one embodiment, the reagent-GABRA3 complex is capable of being detected. Various methods of detection of the reagent-GABRA3 complex are known in the art. In some embodiments, such methods include the use of labels as described herein.

In one embodiment, the ligand is associated with a detectable label or a substrate. The ligand may be covalently or non-covalently joined with the detectable label or substrate. In one embodiment, the comprises a substrate upon which said ligand is immobilized.

For these reagents, the labels may be selected from among many known diagnostic labels, including those described above. Selection and/or generation of suitable ligands with optional labels for use in this invention is within the skill of the art, provided with this specification, the documents incorporated herein, and the conventional teachings of the art. Ligands may be labeled using conventional methods with a detectable substance. Examples of detectable substances include, but are not limited to, the following: radioisotopes (e.g., ³H, ¹⁴C, ³⁵S, ¹²⁵I ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags).

Similarly, the substrates for immobilization may be any of the common substrates, glass, plastic, a microarray, a microfluidics card, a chip or a chamber. The reagent itself may be labeled or immobilized. For example, a ligand or sample may be immobilized on a carrier or solid support which is capable of immobilizing cells, antibodies, etc. Suitable carriers or supports may comprise nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Immobilization typically entails separating the binding agent from any free analytes (e.g. free markers or free complexes thereof) in the reaction mixture.

Still another diagnostic reagent includes a composition or kit comprising at least one reagent that binds to, hybridizes with or amplifies GABRA3. Such diagnostic reagents and kits containing them are useful for the measurement and detection of GABRA3 in the methods described herein for diagnosis/prognosis of cancer or metastasis of cancer. In addition to the reagents above, alternatively, a diagnostic kit thus also contains miscellaneous reagents and apparatus for reading labels, e.g., certain substrates that interact with an enzymatic label to produce a color signal, etc., apparatus for taking blood samples, as well as appropriate vials and other diagnostic assay components.

### II. DIAGNOSTIC METHODS

The compositions described herein are useful in methods of detection, diagnosis, monitoring, and prognosis of breast cancer, or the status of breast cancer, and for the identification of subjects with an increased risk of breast cancer metastasis. In one aspect, a method is provided in which a sample is tested for the presence and/or level of GABRA3. In one embodiment, the presence of GABRA3 in the sample is indicative of breast cancer. In another embodiment, an increase in the level of GABRA3 in the sample as compared to a control, is indicative of breast cancer. In one embodiment, the control is derived from normal breast tissue or cells.

In another aspect, a method of detecting the risk of breast cancer metastasis in a subject is provided. In one embodiment, the method includes measuring the level of GABRA3 in a biological sample from the subject, wherein an increase in the level of GABRA3 in the sample as compared to a control, is indicative of an increased risk of metastasis. In one embodiment, the control is derived from normal breast tissue or cells. In another embodiment the control is derived from a subject (or population of subjects) that have breast cancer that has not metastasized.

In another aspect, a method of detecting the risk of breast cancer metastasis in a subject is provided. In one embodiment, the method includes measuring the level of A-to-I RNA edited GABRA3 in a biological sample from the subject, wherein an decrease in the level of A-to-I RNA edited GABRA3 in the sample as compared to a control, is indicative of an increased risk of metastasis. In one embodiment, the control is a sample derived from a subject (or population of subjects) having breast cancer. Adenosine deaminases acting on RNA (ADARs) can edit nucleotides in the RNA. Specifically, these enzymes can modify a genetically-encoded adenosine (A) into an inosine (I) in double-stranded RNA structures. ADAR editing results in inosine, which replaces the genomically encoded adenosine, and is read by the cellular machinery as a guanosine (G). Thus, sequencing of inosine-containing RNAs results in G where the corresponding genomic DNA reads A. Bazak et al, Published in Advance Dec. 17, 2013, doi: 10.1101/gr.164749.113 Genome Res. 2013, which is incorporated herein by reference. It is demonstrated herein that A-to-I RNA editing of GABRA3 occurs only in non-invasive breast cancer cells, and that edited GABRA3 suppresses breast cancer cell invasion and metastasis.

The presence of GABRA3 in the sample (or a GABRA3-ligand complex) may be detected using any assay format known in the art or described herein. There are a variety of assay formats known to the skilled artisan for using a ligand to detect a target molecule in a sample. (For example, see Harlow and Lane, Antibodies: A Laboratory Manual, Cold Spring Harbor Laboratory, 1988). In general, the presence or absence of GABRA3 in a sample may be determined by (a) contacting the sample with a ligand that interacts with GABRA3; and (b) determining the presence or level of GABRA3 in the sample, wherein the presence of GABRA3 in the sample is indicative of breast cancer or where an increase in the level of GABRA3 in the sample as compared to a control, is indicative of breast cancer. The various assay methods employ one or more of the GABRA3-binding ligands described herein, e.g., polypeptide, polynucleotide, and/or antibody, which detect the GABRA3 protein or mRNA encoding the same (including fragments or portions thereof).

A. Protein Assays

Methods of detection, diagnosis, monitoring, and prognosis of breast cancer, or the status of breast cancer, and for the identification of subjects with an increased risk of breast cancer metastasis by detecting the presence of, or measuring the level of GABRA3 protein, are provided herein. Such methods may employ polypeptides and/or antibodies as described herein.

The particular assay format used to measure the GABRA3 in a biological sample may be selected from among a wide range of immunoassays, such as enzyme-linked immunoassays, sandwich immunoassays, homogeneous assays, immunohistochemistry formats, or other conventional assay formats. One of skill in the art may readily select from any number of conventional immunoassay formats to perform this invention.

Other reagents for the detection of protein in biological samples, such as peptide mimetics, synthetic chemical compounds capable of detecting GABRA3 may be used in other assay formats for the quantitative detection of GABRA3 protein in biological samples, such as high pressure liquid chromatography (HPLC), immunohistochemistry, etc.

B. Nucleic Acid Assays

Methods of detection, diagnosis, monitoring, and prognosis of breast cancer, or the status of breast cancer, and for the identification of subjects with an increased risk of breast cancer metastasis by detecting the presence of, or measuring the level of GABRA3 mRNA, are provided herein. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR.

Such PCR-based method may employ a primer or primer-probe set capable of identifying and/or amplifying a GABRA3 nulceic acid sequence or a portion thereof. An example of a primer set capable of identifying and/or amplifying a GABRA3 nucleic acid sequence or a portion thereof is described in Example 1E. Such primers include GABRA3 Forward-5′-GACCACGCCCAACAAGCT-3′ (SEQ ID NO: 1) and Reverse-5″-AGCATGAATTGTTAACCTCATTGTATAGA-3′ (SEQ ID NO: 2). Other suitable primers can be designed by the person of skill in the art and/or obtained commercially based on the GABRA3 nucleic acid sequence. Such sequences are known in the art and can be found, e.g., at NCBI Reference Sequence: NM_000808.3.

An example of a method to quantify the A-to-I edited GABRA3 as compared to the unedited GABRA3 is described in Example 1H and in the art. See, e.g., Nicholas et al, Age-related gene-specific changes of A-to-I mRNA editing in the human brain, Mech Ageing Dev. 2010 June; 131(6): 445-447 which is incorporated herein by reference.

Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

The diagnostic methods described herein can employ contacting a patient's sample with a diagnostic reagent, as described above, which forms a complex or association with GABRA3 in the patients' sample. Detection or measurement of the sample GABRA3 may be obtained by use of a variety of apparatus or machines, such as computer-programmed instruments that can transform the detectable signals generated from the diagnostic reagents complexed with the GABRA3 in the biological sample into numerical or graphical data useful in performing the diagnosis. Such instruments may be suitably programmed to permit the comparison of the measured GABRA3 in the sample with the appropriate reference standard and generate a diagnostic report or graph.

The selection of the polynucleotide sequences, their length and labels used in the composition are routine determinations made by one of skill in the art in view of the teachings of which genes can form the gene expression profiles suitable for the diagnosis and prognosis of breast cancer. For example, useful primer or probe sequences can be at least 8, at least 10, at least 15, at least 20, at least 30, at least 40 and over at least 50 nucleotides in length. For example, such probes and polynucleotides can be complementary to portions of mRNA sequences encoding GABRA3. The probes and primers can be at least 70%, at least 80%, at least 90%, at least 95%, up to 100% complementary to sequences encoding.

In any of the methods described herein, in one embodiment, the sample comprises breast cells. Such sample may be derived from a tissue biopsy.

In some of the methods described herein, a control level is used as a reference point. The control level can be any of those described herein. In one embodiment, the control level is the level obtained from an individual, or a population of individuals, who are healthy (i.e., who do not have breast cancer). In another embodiment, the control level is the level obtained from an individual, or a population of individuals, who have breast cancer that has not metastasized.

### II. TREATMENT METHODS

In another aspect, methods of treating breast cancer are provided. In one embodiment, the method of treating breast cancer includes inhibiting the action of GABA in a subject. In one embodiment, the method includes treating the subject with a GABAA receptor antagonist. In another embodiment, the method includes reducing the level of expression or activity of GABRA3 in the subject.

In another aspect, a method of reducing migration and/or invasion of breast cancer cells is provided. In on embodiment, the method includes inhibiting the action of GABA in a subject. In one embodiment, the method includes treating the subject with a GABAA receptor antagonist. In another embodiment, the method includes reducing the level of expression or activity of GABRA3 in the subject.

In yet another aspect, a method of treating breast cancer includes detecting the presence or measuring the level of GABRA3 in a biological sample from a subject and treating the subject with a reagent that inhibits the action of GABA when GABRA3 is detected in the sample or when there is an increase in the level of GABRA3 in the sample as compared to a control sample from a healthy subject. In one embodiment, the method includes treating the subject with a GABAA receptor antagonist. In another embodiment, the method includes reducing the level of expression or activity of GABRA3 in the subject. The GABRA3 may be detected using the reagents and/or methods described herein.

A number of different classes of pharmacological agents exert their effects on the GABAA receptor by binding to recognition sites that are distinct from the endogenous ligand (GABA) binding site. In this regard, the benzodiazepine recognition site is the best understood based upon not only the proven clinical efficacy of compounds acting at this site but also the availability of pharmacological tool compounds as well as genetically modified mice. However, other binding sites, including the GABA binding site, the benzodiazepine binding site, the neurosteroid binding site, convulsant binding site, barbituate binding site, the subunit binding sites, and the ion channel pore are contemplated targets for the GABAA receptor antagonists, as discussed herein. Such agents are termed “GABA antagonists” or “GABAA receptor antagonists” and include all agents which either directly or allosterically modulate the inhibitory function of GABA. In one embodiment, this includes compounds such as flumazenil, which bind with high affinity but do not affect GABA-induced chloride currents, exert no physiological effect on the GABAA receptor but can block, or antagonise, the effects of benzodiazepine site agonists or inverse agonists. These compounds are sometimes described as benzodiazepine site antagonists. In another embodiment, the GABAA receptor antagonists described herein include benzodiazepine site “inverse agonists”, which reduce GABA-mediated chloride flux.

Many suitable GABAA receptor antagonists are known in the art. Such antagonists include, without limitation, bicuculline, gabazine, Iso-THAZ, flumazenil, and DMCM. Other antagonists include, without limitation, allopregnanolone, alphaxalone, 3α,5α-THDOC, ganaxolone, org21465, and 17PA. Other antagonists include, without limitation, picrotoxinin, picrotin, picrotoxin, TBPS, and PTZ. Yet further antagonists include, without limitation, pentobarbital, methodhexital, phenobarbital, and secobarbital. Other antagonists include, without limitation, loreclezole, etomitade and propofol. Yet other antagonists include, without limitation, thiocolchicoside, pentetrazol, and topiramate. Further antagonists include, without limitation, L-838417, TPA003, MRK-529, and NS11394. Other antagnonists are known in the art or may be developed in the future. See, Atack 2010, cited above. In one embodiment, the GABAA receptor antagonist is flumazenil. In another embodiment, the GABAA receptor antagonist is picrotoxin. In another embodiment, the GABAA receptor antagonist is pentetrazol. In another embodiment, the GABAA receptor antagonist is topiramate.

The dosages and treatment regimens utilizing GABAA receptor antagonists can be determined by the person of skill in the art. Certain of the GABAA receptor antagonists are approved for use for the treatment of other conditions, and thus dosages and prescribing information is known. For example, in the case of flumazenil, in one embodiment, a dosage of from about 10 nM to about 10 μM is provided to treat breast cancer. In another embodiment, a dosage of 0.4 mg-1.0 mg IV is provided.

According to these methods, one or more of the GABAA receptor antagonists, noted above, are administered prior to or during a course of chemotherapy or radiation to reduce the size of an existing primary tumor. In another embodiment, the methods involve administering a dose of the GABAA receptor antagonist prior to or during surgery for tumor removal. In still another embodiment, the methods comprise administering GABAA receptor antagonist after surgery. In yet a further embodiment, the methods involve administering GABAA receptor antagonist prior to or during a second or repeated course of chemotherapy or radiation. In certain embodiments, the second or repeated course is post-surgery. Still further embodiments of the methods described herein include administering a continuous course of a dose of a GABAA receptor antagonist to a subject in need thereof. Such courses of therapy may be repeated. Still a further embodiment of the method includes administering an intermittent course of a dose of GABAA receptor antagonist to a subject in need thereof. Other regimens may be selected by the attending physician based upon the condition and responsiveness of the subject to the therapy.

The dosage required for the one or more GABAA receptor antagonists depends primarily on factors such as the condition being treated, the age, weight and health of the patient, and may thus vary among patients. The effective dosage of each active component is generally individually determined, although the dosages of each compound can be the same. In one embodiment, the small molecule dosage is about 1 μg to about 1000 mg. In one embodiment, the effective amount is about 0.1 to about 50 mg/kg of body weight including any intervening amount. In another embodiment, the effective amount is about 0.5 to about 40 mg/kg. In a further embodiment, the effective amount is about 0.7 to about 30 mg/kg. In still another embodiment, the effective amount is about 1 to about 20 mg/kg. In yet a further embodiment, the effective amount is about 0.001 mg/kg to 1000 mg/kg body weight. In another embodiment, the effective amount is less than about 5 g/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg. However, the effective amount of the GABAA receptor antagonist(s), as well as dosages different than that used for brain-related conditions, can be determined by the attending physician and depends on the condition treated, the compound administered, the route of delivery, age, weight, severity of the patient's symptoms and response pattern of the patient.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays (such as those described in the examples below) and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

One or more of the GABAA receptor inhibitors discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

The therapeutic compositions administered by these methods are administered directly into the environment of the targeted cell undergoing unwanted proliferation, e.g., a cancer cell or targeted cell (tumor) microenvironment of the patient. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically.

These therapeutic compositions, i.e., GABAA receptor antagonists, may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

Because the compositions do not have to cross the blood-brain-barrier, alternate compositions can be provided which do not meet the characteristics required to do so, yet still inhibit the action of GABA in breast cells. Thus, in yet another aspect, a method of screening molecules for use in cancer therapy comprises contacting a mammalian cancer or tumor cell culture which express GABRA3 with a potential therapueutic molecule, e.g., a small molecule, peptide, nucleotide sequence, intracellular antibody or the like; and culturing the cell. The culture is then tested for inhibition of celluar migration. An example of a celluar migration assay is described in Example 1E. Other methods are known in the art. If cellular migration is decreased as compared to a control, the molecule has an anti-tumor or anti-cancer effect, or prevents or reduces cancer metastasis. The level of cellular migration in the test cell culture an be compared to the level of celluar migration in untreated cancer/tumor cell cultures.

In another aspect, methods of treating breast cancer involve inhibiting, suppressing or down-regulating the expression or overexpression of GABRA3 in the subject's cancer or tumor cells. These methods can employ a variety of type of reagents to effect the inhibition, suppression or down-regulation. More specifically, these methods in certain embodiments, involve administering to a subject a therapeutically effective dose of a reagent that binds to or interacts with GABRA3.

As one example, a reagent for such administration can be an antibody or antibody fragment specific for GABRA3. As used herein, the term “antibody,” refers to an immunoglobulin molecule which is able to specifically bind to GABRA3.

In a further embodiment, a suitable reagent for administration in these methods includes a small molecule that binds to the three dimensional structure of GABRA3. In still another embodiment, a suitable reagent for administration in these methods includes nucleic acid sequences or molecules that bind, hybridize to, or amplify a target sequence, e.g., GABRA3. The term nucleic acid sequence as used herein includes unmodified RNA or DNA or modified RNA or DNA or cDNA. In certain embodiments, nucleic acid sequences or molecules are single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. The term includes DNAs (including cDNAs) and RNAs that contain one or more modified bases. In general, the term nucleic acid sequences or molecules embraces all chemically, enzymatically and/or metabolically modified forms of unmodified polynucleotides, oligonucleotides, as well as the chemical forms of DNA and RNA characteristic of viruses and cells, including simple and complex cells. These sequences can be synthesized by chemical methods, prepared by in vitro recombinant DNA-mediated techniques and by expression of DNAs in cells and organisms.

In one embodiment, a reagent for these methods is an “antisense” nucleotide sequence or a small nucleic acid molecule having a complementarity to a target nucleic acid sequence, e.g., a nucleic acid sequence that binds or hybridizes to a nucleic acid sequence encoding GABRA3 or from which GABRA3 is transcribed. In one embodiment, such binding or hybridizing reduces the expression or silences the transcription of GABRA3.

As one example, a suitable reagent is a short nucleic acid molecule capable of inhibiting or down-regulating GABRA3 gene or protein expression. Typically, short interfering nucleic acid molecules are composed primarily of RNA, and include siRNA or shRNA, as defined below. A short nucleic acid molecule may, however, include nucleotides other than RNA, such as in DNAi (interfering DNA), or other modified bases. Thus, the term “RNA” as used herein means a molecule comprising at least one ribonucleotide residue and includes double stranded RNA, single stranded RNA, isolated RNA, partially purified, pure or synthetic RNA, recombinantly produced RNA, as well as altered RNA such as analogs or analogs of naturally occurring RNA. In one embodiment the short nucleic acid molecules of the present invention is also a short interfering nucleic acid (siNA), a short interfering RNA (siRNA), a double stranded RNA (dsRNA), a micro RNA (μRNA), and/or a short hairpin RNA (shRNA) molecule. The short nucleic acid molecules can be unmodified or modified chemically. Nucleotides of the present invention can be chemically synthesized, expressed from a vector, or enzymatically synthesized. An example of shRNA knockdown of GABRA3 is described in the Examples, e.g., Example 1B.

In some embodiments, the short nucleic acid comprises between 18 to 60 nucleotides. In another embodiment, the short nucleic acid molecule is a sequence of nucleotides between 25 and 50 nucleotides in length. In still other embodiments, the short nucleic acid molecule ranges up to 35 nucleotides, up to 45, up to 55 nucleotides in length, depending upon its structure. These sequences are designed for better stability and efficacy in knockdown (i.e., reduction) of GABRA3 gene expression. In one embodiment, the nucleic acid molecules described herein comprises 19-30 nucleotides complementary to a GABRA3 nucleic acid sense sequence, particularly an open reading frame of GABRA3. In one embodiment, the nucleic acid molecules described herein comprises 19-30 nucleotides complementary to a GABRA3 antisense nucleic acid sequence strand.

In one embodiment, a useful therapeutic agent is a small interfering RNA (siRNA) or a siRNA nanoparticle. siRNAs are double stranded, typically 21-23 nucleotide small synthetic RNA that mediate sequence-specific gene silencing, i.e., RNA interference (RNAi) without evoking a damaging interferon response. siRNA molecules typically have a duplex region that is between 18 and 30 base pairs in length. GABRA3 siRNAs are designed to be homologous to the coding regions of GABRA3 mRNA and suppress gene expression by mRNA degradation. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. The design of si/shRNA preferably avoids seed matches in the 3′UTR of cellular genes to ensure proper strand selection by RISC by engineering the termini with distinct thermodynamic stability. A single siRNA molecule gets reused for the cleavage of many target mRNA molecules. RNAi can be induced by the introduction of synthetic siRNA. In one embodiment, a siRNA molecule of the invention comprises a double stranded RNA wherein one strand of the RNA is complimentary to the RNA of GABRA3. In another embodiment, a siRNA molecule of the invention comprises a double stranded RNA wherein one strand of the RNA comprises a portion of a sequence of RNA having GABRA3 sequence. Synthetic siRNA effects are short lived (a few days) probably because of siRNA dilution with cell division and also degradation.

In another aspect, a method of treating breast cancer includes increasing the level of A-to-I RNA edited GABRA3. Methods of increasing the level of A-to-I RNA edited GABRA3 are known in the art and include provision of the A-to-I edited mRNA or coding sequence to the subject. Various methods of providing the A-to-I edited mRNA or coding sequence to the subject are known in the art and include, without limitation, the use of viral vectors. In one embodiment, the nucleic acid sequences encoding A-to-I RNA edited GABRA3 are engineered into any suitable genetic element, e.g., naked DNA, phage, transposon, cosmid, RNA molecule (e.g., mRNA), episome, etc., which transfers the A-to-I RNA edited GABRA3 sequences carried thereon to a host cell, e.g., for generating nanoparticles carrying DNA or RNA, viral vectors in a packaging host cell and/or for delivery to a host cell in a subject.

In another aspect, the use of a composition comprising a reagent that decreases GABRA3 levels of expression or activity, for the manufacture of a medicament for use in treating breast cancer ir provided. In one embodiment, the reagent that decreases GABRA3 levels is a GABAA receptor antagonist.

In another aspect, the use of a composition comprising a reagent that inhibits the action of GABA, for the manufacture of a medicament for use in treating breast cancer ir provided. In one embodiment, the reagent that inhibits the action of GABA levels is a GABAA receptor antagonist.

### II. EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

Metastasis is a critical factor affecting breast cancer patient survival. In order to identify novel molecules in metastatic process, a bioinformatics analysis was performed using The Cancer Genome Atlas (TCGA) breast cancer data. identified 41 genes were identified whose expression was inversely correlated with survival. As demonstrated in the examples below, high expression of GABA_{A} receptor GABRA3 was found to be inversely correlated with breast cancer survival in TCGA data. It was found that GABRA3, normally exclusively expressed in normal adult brain tissues, is also expressed in breast cancer cells. The inventors demonstrate that GABRA3 activates the AKT pathway to promote cell migration, invasion, and metastasis in vitro and in mouse models and that GABRA3 knockdown reduces cell invasion and metastasis. Interestingly, we demonstrate A-to-I RNA editing of GABRA3 only in non-invasive breast cancer cells and show that edited GABRA3 suppresses breast cancer cell invasion and metastasis. A-to-I editing of GABRA3 reduces expression on cell surface and also suppresses AKT activation required for cell migration and invasion.

### Example 1: Materials and Methods

A. TCGA Data Analysis

RNA-seq of TCGA breast cancer and normal breast tissue data set were compared using the EdgeR method to find genes significantly dysregulated in cancer. For the cancer group, we also performed Cox regression analysis in order to find genes significantly associated with breast cancer survival. Genes were identified that satisfied the following 4 conditions: (1) genes were significantly differentially expressed in samples from breast cancer compared to samples from normal breast tissue (FDR<5%); (2) the difference in expression was at least 5 fold; (3) which were significantly associated with survival (p<0.05); and (4) for which the direction of gene expression difference aligned with the direction of survival, that is, genes upregulated in cancer had to have their higher expression be associated with poor survival, and vice versa.

B. Lentivirus Transfection and Transduction

To generate MCF7 cells stably overexpressing GABRA3, full-length human GABRA3 was amplified by PCR with F-5′-ATGATAATCA CACAAACAAG TCACTG-3′ and R-5′-CTACTGTTTGCGGATCATGCC-3′ primers and cloned into a lentiviral vector. Lentivirus was produced by co-transfecting subconfluent human embryonic kidney (HEK) 293T cells with GABRA3 expression plasmid or vector along with packaging plasmids pMDLg/pRRE and RSV-Rev) using Lipifectamine 2000 as previously described (19, 20). Infectious lentiviruses were collected 48 h after transfection, centrifuged to remove cell debris and filtered through 0.45 μm filters (Millipore). MCF7 cells were transduced with the GABRA3 lentivirus. Efficiency of overexpression was determined by real-time PCR. MDA-MB-436 expressing GABRA3 shRNA (Sigma) or control shRNA (Sigma) were established using vector based shRNA technique. The lentiviruses were processed as described above and transduced into MDA-MB-436 cells. The knockdown efficiency was determined real time PCR. RNA edited GABRA3 (A-I) was generated using the QuikChange II XL Site-Directed Mutagenesis Kit (Agilent), cloned into a lentivirus vector and transduced MCF7 expressing GABRA3 or MDA-MB-436 cells.

C. Mammary Fat Pad Injections

The MCF7 or MDA-MB-436 human breast cancer cell lines stably expressing Firefly Luciferase gene with GABRA3, or GABRA3 shRNA, or control vector, or RNA edited GABRA3 (A-to-I) were routinely maintained at 37° C. in a humidified atmosphere of 5% CO₂ and 95% air in DMEM medium supplemented with 10% fetal bovine serum (FBS). For orthotopic injections, MCF7 (7×10⁶ cells/mouse) were transplanted into the mammary fat pads of the female SCID mice (6-8 weeks old). A slow-release pellet of 17β-estradiol (1.7 mg, 90-day release; Innovative Research of America, Sarasota, Fla.) was implanted subcutaneously in the dorsal interscapular region before the transplantation of MCF7 cells. MDA-MB-436 (1×10⁶) were suspended in 100 μL of PBS and injected in the lateral tail vein of 6-8 weeks old NOD/SCID mice. Mice bearing luciferase positive tumors were imaged by IVIS 200 Imaging system (Xenogen Corporation, Hopkinton, Mass.). Bioluminescent flux (Photons/sec/sr/cm²) was determined for the primary tumors and lung metastasis. Animal experiment protocols were approved by the Institutional Animal Care and Use Committee (IACUC) of the Wistar Institute. Animal procedures were conducted in compliance with the IACUC.

D. Transwell Migration and Invasion Assay

In vitro cell migration assays were performed as described previously (21, 22) using Trans-well chambers (8 μM pore size; Costar). Cells were allowed to grow to subconfluency (˜75-80%) and were serum-starved for 24 h. After detachment with trypsin, cells were washed with PBS, resuspended in serum-free medium and 250 μl cell suspensions (2×10⁵ cells ml−1) was added to the upper chamber. Complete medium was added to the bottom wells of the chambers. The cells that had not migrated were removed from the upper face of the filters using cotton swabs, and the cells that had migrated to the lower face of the filters were fixed with 5% glutaraldehyde solution and stained with 0.5% solution of Toluidine Blue in 2% sodium carbonate. Images of three random ×10 fields were captured from each membrane and the number of migratory cells was counted. The mean of triplicate assays for each experimental condition was used. Similar inserts coated with Matrigel were used to determine invasive potential in the invasion assay. To assess the effect of AKT kinase inhibitor MK2206 on MCF7-GABRA3 cell migration and invasion, assays were performed as described above in the presence of different concentrations of the inhibitor or control DMSO.

E. RNA Isolation, Reverse Transcription and Real-Time PCR Analysis.

Total RNA was extracted from cell lines, using Trizol total RNA isolation reagent (Invitrogen), according to the manufacturer's specifications and treated with Turbo DNase (Ambion). cDNA was synthesized from total RNA (0.5 μg) using random hexamers with TaqMan cDNA Reverse Transcription Kit (Applied Biosystems). Gene primers (GABRA3 F-5′-GACCACGCCCAACAAGCT-3′; R-5″-AGCATGAATTGTTAACCTCATTGTATAGA-3′; GAPDH-F-5′-GAAGGTGAAGGT CGGAGTCAAC; R-5′-CAGAGTTAAAAGCAGCCCTGGT) were designed using Primer Express v3.0 Software and real-time PCR was performed using SYBR Select Master Mix (Applied Biosystems). All reactions were carried out on the 7500 Fast Real Time PCR system (Applied Biosystem). The average of three independent analyses for each gene and sample was calculated using the AA threshold cycle (Ct) method and was normalized to the endogenous reference control gene GAPDH.

F. Western Blotting

Standard methods were used for western blotting. Cells were lysed in lysis buffer and total protein contents were determined by the Bradford method. 30 μg of proteins were separated by SDS-PAGE under reducing conditions and blotted onto a polyvinylidene difluoride membrane (Millipore). Membranes were probed with specific antibodies Blots were washed and probed with respective secondary peroxidase-conjugated antibodies, and the bands visualized by chemoluminescence (Amersham Biosciences). The following antibodies were used: Rabbit polyclonal GABRA3 (Santa Cruz Biotechnology), mouse monoclonal ADAR1 (Santa Cruz Biotechnology), Rabbit polyclonal AKT, pAKT (Cell Signaling Technology), mouse monoclonal β-actin (Sigma-Aldrich), and secondary peroxidase conjugated (GE healthcare).

G. Flow Cytometry

For surface FACS (in order to detect surface protein expression), cells at 80% confluence were washed with PBS and harvested with Versene (Sigma-Aldrich) for 15 min at 37° C. Cells were centrifuged, resuspended in FACS buffer (0.5% BSA in PBS) and incubated with human GABRA3 (Antibodies online) or IgG isotype control (Life technologies) antibodies at a 1:50 dilution, for 60 min at 4° C., then washed twice in FACS buffer. For detection of GABRA3, cells were incubated with goat anti-rabbit Alexa Fluor 488 (Invitrogen, A-11008) for 30 min at 4° C., and washed twice in FACS buffer. Cells were analyzed on a BD FACS Calibur cell analyser (BD Biosciences) and FACS data were computed using the FLOJo software.

H. RNA Isolation and GABRA3 RNA Editing Analysis

Total RNA was extracted from cell lines, using Trizol total RNA isolation reagent (Invitrogen), according to the manufacturer's specifications and treated with Turbo DNase (Ambion). cDNA was synthesized from 1 μg of total RNA with GABRA3 gene specific primer 5′TTCAGTGTCCTTGGCCAGGTT 3′ using SuperScript III reverse transcriptase (Invitrogen). We aimed for high sequence quality thus performed nested PCR. PCR primers were designed with in edited site to generate amplicons of the expected size only from mRNA but not from genomic DNA. 1^{st} PCR product was amplified using first-strand cDNA templates and GABRA3 forward (5′-TCACAAGTGTCGTTCTGGCTCA-3′) and reverse primer (5′-TTCAGTGTC CTTGGCC AGGTT3′). 2^{nd} PCR was performed using first PCR product and GABRA3 forward (5′-CAAGTGTCGTTCTGGCTCAACA-3′) and reverse (5′-AGTGTCCTTG GCCAGGTTGAT-3′) primers. The resulting PCR fragments were purified using QIAquick gel extraction kit (Qiagen). The level of RNA editing is assessed by direct sequencing of each purified PCR product using the reverse primer used for 2^{nd} PCR amplification. The percentage of A-to-I editing was determined by dividing the height of G peak at the editing site by the height of A peak plus G peak from the sequencing chromatogram.

### Example 2: Identification of GABRA3 in Breast Cancer Progression

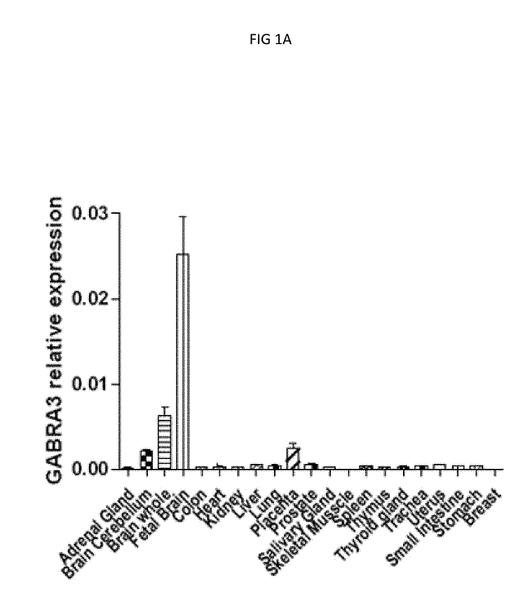

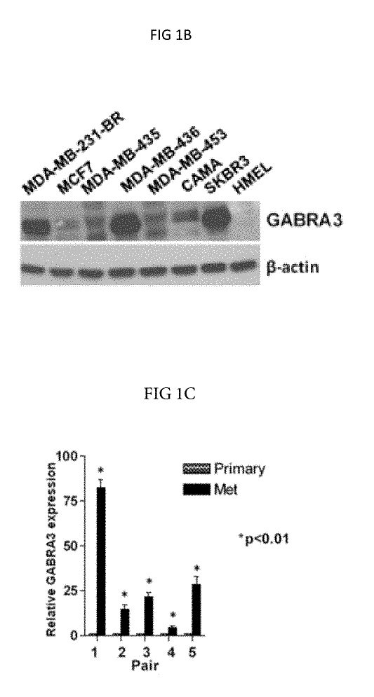

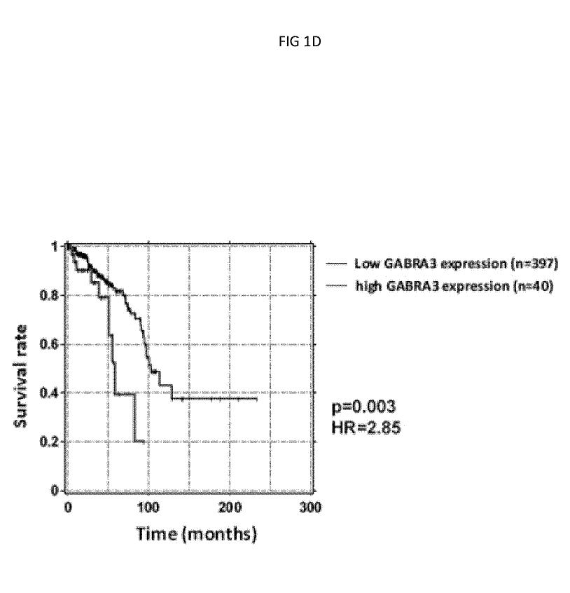

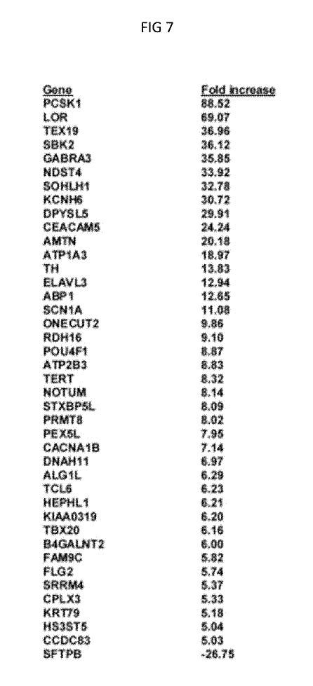

To identify genes that are critical for breast cancer progression, we analyzed the RNA-seq of the TCGA breast cancer and normal breast tissue data set as well as data associated with survival (see Materials and Methods). We identified 41 genes that met the four conditions described in Materials &Methods (FIG. 7). Among these genes, up-regulation of 40 genes of them and down-regulation of one (SFTBP) is associated with poor survival. Among the genes for which overexpression is associated with poor prognosis, many had been previously shown to be significantly upregulated in cancer. For example, up-regulation of telomerase expression has been shown to be critical in cancer development in multiple cancer types (23). Several transcription factors including ONECUT2, POU4F1, and NOTUM, have also been previously been shown to promote tumorigenesis (24-26). We chose to focus on the chloride channel protein GABRA3 for several reasons: it is highly expressed in cancer tissues but not in normal breast tissues; it is a cell surface molecule, a potential drug-targetable protein; therapeutics targeting GABRA3 are already used in the clinics for other purposes. We first determined the expression pattern of GABRA3 in a panel of normal human tissues and found that it was expressed much more strongly in adult brain tissues than in other adult organs (FIG. 1A). Immunoblotting of GABRA3 in human breast cancer cell lines indicates that it was expressed at various levels in breast cancer cell lines but not expressed in normal human epithelial cell HMEL (FIG. 1B). In paired human breast cancer samples, GABRA3 expression was higher in the metastatic samples than that of primary breast cancer samples (FIG. 1C). The TCGA survival data indicated that higher GABRA3 expression correlated with worse survival (FIG. 1D).

### Example 3: GABRA3 Promotes Breast Cancer Invasion and Metastasis In Vitro and in Mouse Models

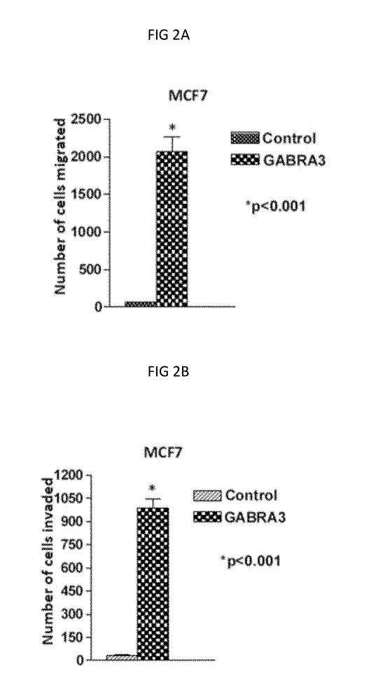

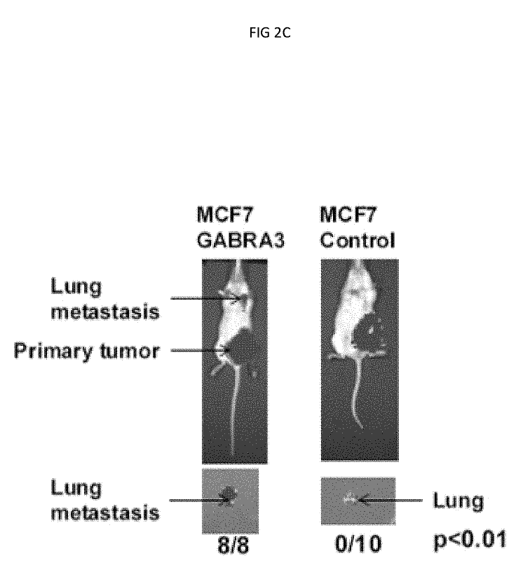

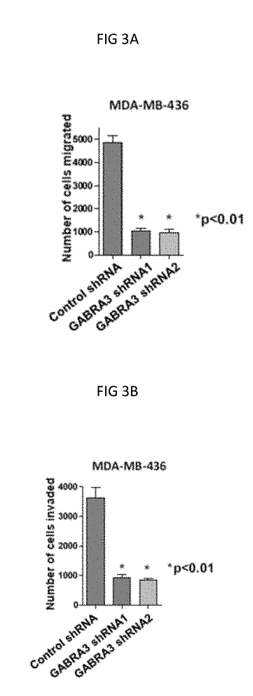

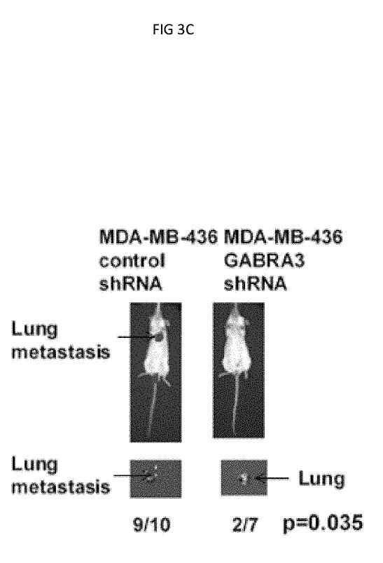

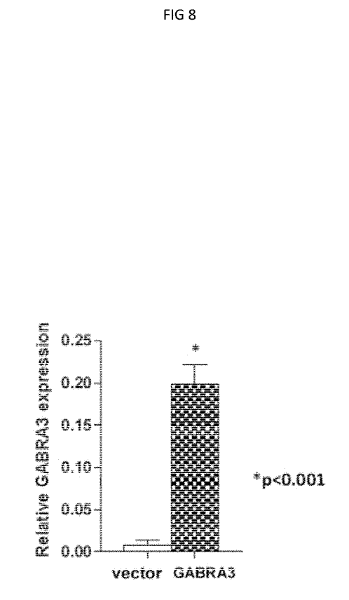

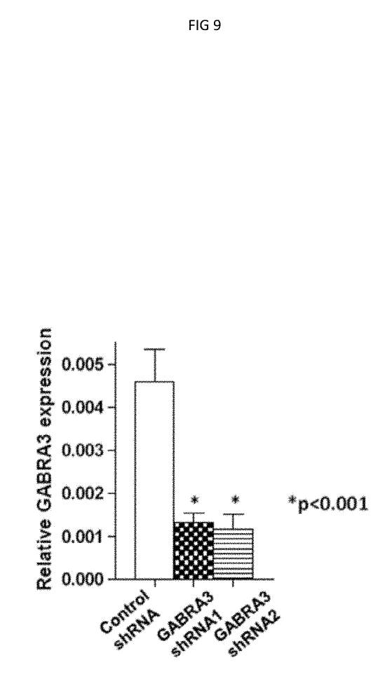

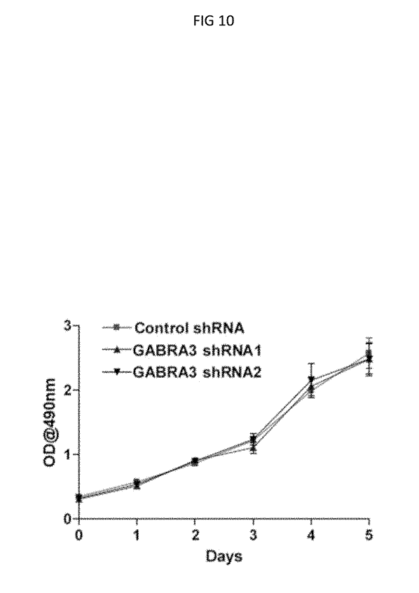

As GABRA3 appeared to be more highly expressed in metastatic tissues than in primary tumors, we assessed the contribution of GABRA3 to cell migration and invasion. We introduced GABRA3 into human breast cancer MCF7 cells which express endogenous GABRA3 at low level and subject these cells to migration and invasion assays. The expression of GABRA3 was confirmed by real time PCR (FIG. 8). MCF7 cells expressing GABRA3 significantly increased migration (FIG. 2A) and invasion (FIG. 2B) capabilities when compared with cells expressing a control vector. We then measured the metastasis-promoting activity of GABRA3 in vivo. Luciferase-tagged MCF-7 cells expressing GABRA3 or a control vector were transplanted into mice mammary fat pads. Lung metastasis were developed following transplantation in all the mice injected with cells expressing GABRA3, whereas no metastasis was observed in any of the mice injected with cells expressing a control vector (FIG. 2C), suggesting that GABRA3 expression is sufficient for metastases promotion. To determine whether GABRA3 is required for breast cancer metastasis, we introduced short hairpin RNA constructs into human breast cancer MDA-MB-436 cells which express high level of GABRA3. Knockdown of GABRA3 was confirmed by real-time PCR (FIG. 9). Knockdown of GABRA3 in MDA-MB-436 cells does not affect cell proliferation (FIG. 10). Strikingly, knockdown of GABRA3 significantly reduced the migration (FIG. 3A) and invasion (FIG. 3B) capabilities of MDA-MB-436 cells. Luciferase-tagged MDA-MB-436 cells expressing GABRA3 shRNAs or a control shRNA were transplanted into mice. Nine out of ten mice injected with cells expressing a control shRNA developed lung metastasis whereas only 2 out 7 mice injected with cells expressing a GABRA3 shRNA developed metastasis (FIG. 3C). Taken together, these results suggested that GABRA3 is both sufficient and required for breast cancer metastasis.

### Example 4: RNA-Edited GABRA3 Suppresses Breast Cancer Invasion and Metastasis In Vitro and in Mouse Models

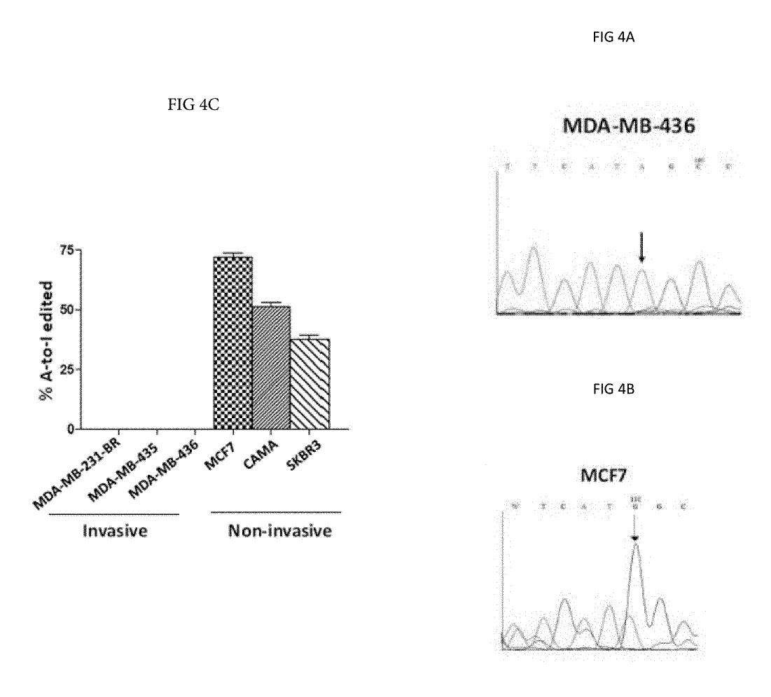

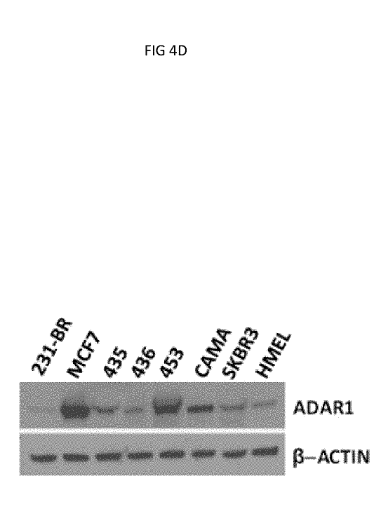

GABRA3 has been shown to be A-to-I RNA-edited in the human brain (18). Whether this is the case for GABRA3 as expressed in human breast cancer tissue was not previously known. We explored this question by directly sequencing GABRA3 mRNA in human breast cancer cells. We found that RNA editing of GABRA3 existed in non-invasive MCF7, CAMA and SKBR3 breast cancer cells (FIG. 4A-C). Invasive cell lines MBA-MD-231, MBA-MD-435, and MBA-MD-436 cells did not express RNA-edited GABRA3 (FIG. 4A-C). Two enzymes responsible for adenosine-to-inosine editing are ADAR1 and ADAR2 (10). We then determined the expression of ADAR1 and ADAR2 in these cell lines. We found that ADAR1 was expressed in all the breast cancer cell lines we tested along with normal human epithelial cell HMEL (FIG. 4D), but ADAR2 was not expressed in any of the cell lines (data not shown).

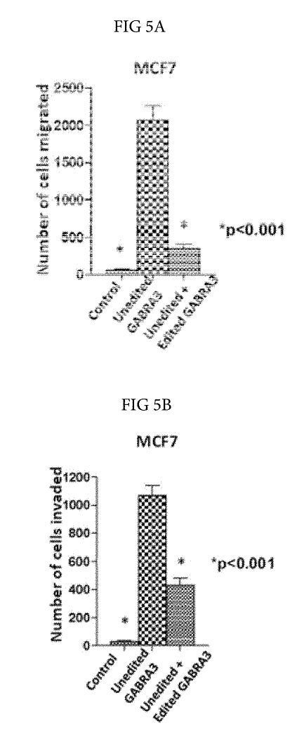

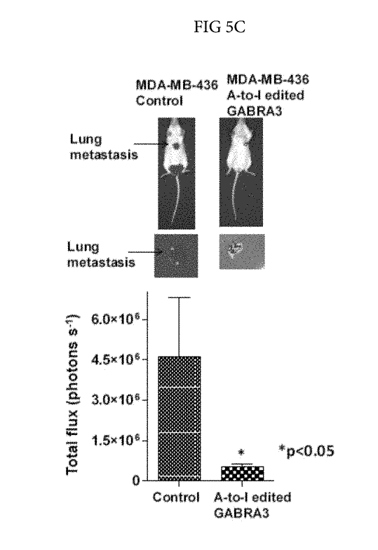

To determine the functions of A-to-I edited GABRA3 in breast cancer cells, we introduced RNA-edited GABRA3 into MDA-MB-436 cells that endogenously express only unedited GABRA3, and subjected these cells to migration and invasion assays. The migration and invasion capabilities of the cells expressing edited GABRA3 message were significantly reduced when compared with the cells expressing a control vector (data not shown). Similarly phenotypes were observed in MCF7 cells. Stable expression of unedited GABRA3 in MCF7 cells promoted cell migration (FIG. 5A) and invasion (FIG. 5B). In contrast, the expression of edited GABRA3 in these cells reversed the migratory (FIG. 5A) and invasive phenotypes (FIG. 5B). To determine RNA-edited GABRA3 had similar effects in vivo, luciferase-tagged MDA-MB-436 cells expressing A-to-I edited GABRA3 or a control vector were transplanted into the mammary fat pads of mice. The luciferase signal indicating lung metastasis was significantly reduced in mice transplanted with the cells expressing RNA-edited GABRA3 compared to mice with the cells expressing a control vector (FIG. 5C). These results suggested that A-to-I edited GABRA3 had an opposing function compared to unedited GABRA3 and suppressed rather than induced invasion and metastasis in breast cancer.

### Example 5: RNA-Edited GABRA3 Reduces GABRA3 Protein Expression on the Cell Surface and Suppresses AKT Activation

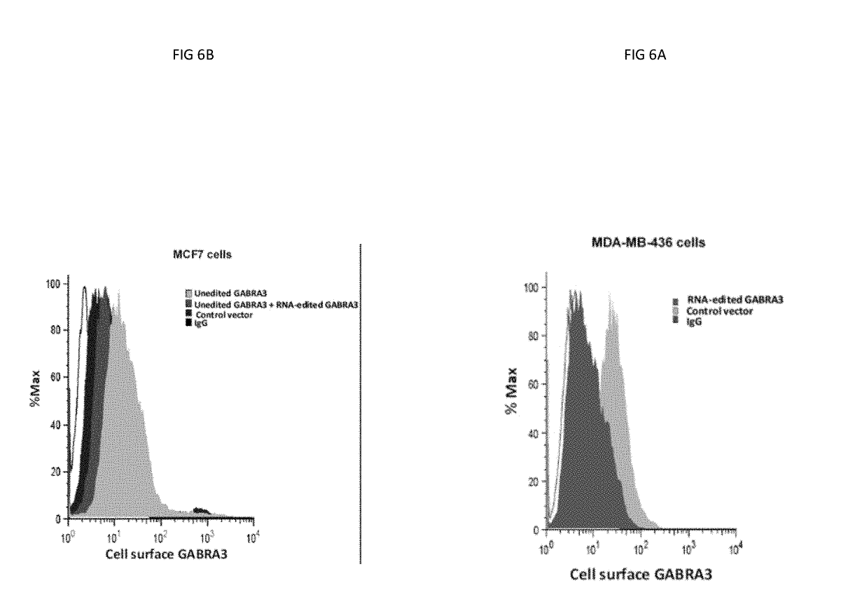

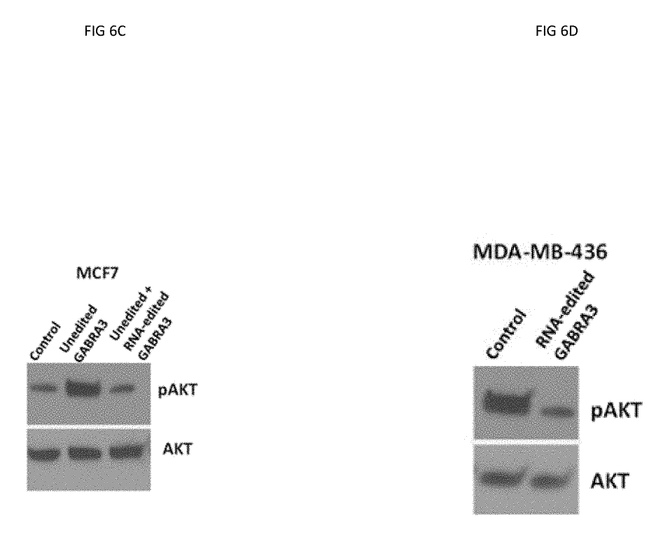

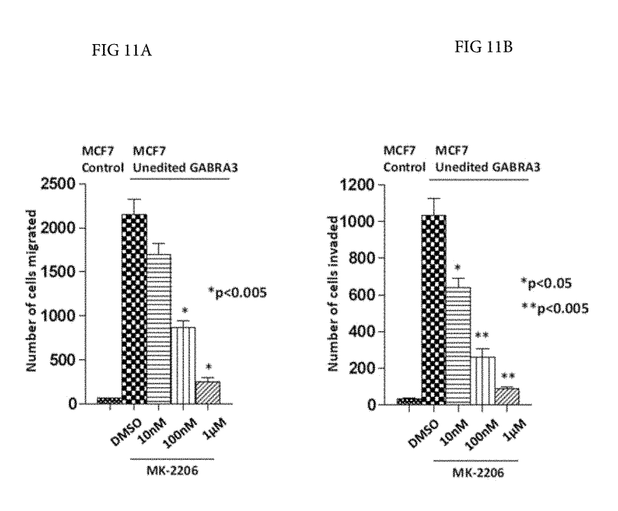

It has previously been shown that A-to-I edited GABRA3 affected the intracellular trafficking of GABRA3 (18), which is critical for the localization of GABRA3 on cell membrane. We determined the expression of GABRA3 on cell surface of breast cancer cells using FACS analysis. MDA-MB-436 cells expressing RNA-edited GABRA3 or a control vector and MCF7 cells expressing unedited GABRA3, or unedited GABRA3 plus RNA-edited GABRA3, or a control vector were used in the FACS analysis. FACS analysis indicated that A-to-I edited GABRA3 reduces the expression of GABRA3 on cell surface in MDA-MB-436 cells (FIG. 6A). Overexpression of unedited GABRA3 in MCF7 cells increased the expression of GABRA3 on cell surface (FIG. 6B), expression of RNA-edited GABRA3 reversed this phenotype (FIG. 6B). The downstream signaling pathways that mediate the functions of GABRA3 in tumor invasion and metastasis are unknown. Since AKT pathway is critical in both breast cancer metastasis and therapy resistance (27-29), we determined the effect of GABRA3 on AKT activation and whether AKT pathway mediates GABRA3 functions in migration and invasion. MCF7 cells stably expressing unedited GABRA3 were treated with a specific pan-AKT inhibitor MK-2206 and cells were subjected to migration and invasion assays. MK-2206 inhibited cell migration and invasion in a dose-dependent manner (FIG. 11) and reversed the migratory and invasive phenotypes of GABRA3, indicating AKT mediated the promoting functions of GABRA3 in migration and invasion. Ectopic expression of RNA edited GABRA3 reduced the phosphorylated AKT but did not affect total AKT in MDA-MB-436 cells (FIG. 6C). Overexpression of unedited GABRA3 increased the expression of phosphorylated AKT in MCF7 cells (FIG. 6D). Overexpression of A-to-I edited GABRA3 reversed the AKT activation caused by unedited GABRA3 overexpression (FIG. 6D). These results suggested that GABRA3 activated AKT pathway, and RNA-edited GABRA3 reduced cell surface GABRA3 and suppressed AKT activation.

### Example 6: Treatment of Breast Cancer Cells with GABRA3 Inhibitors

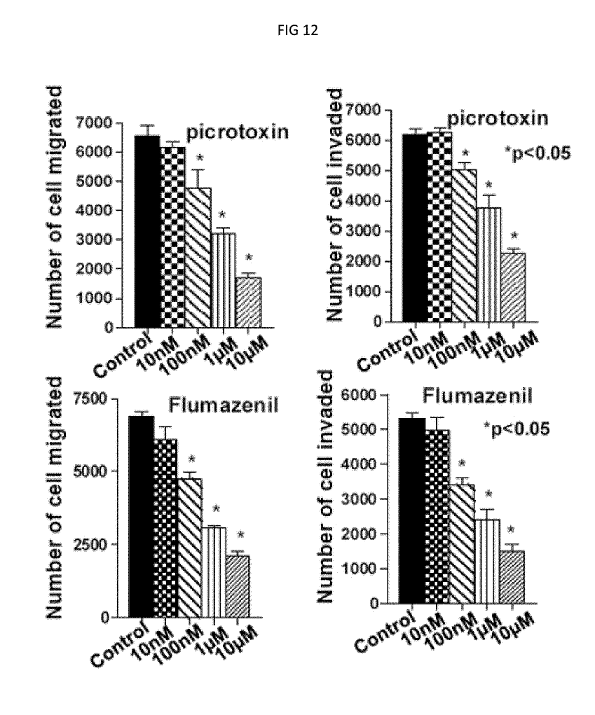

MDA-MB-436 cells were treated with GABRA3 inhibitors picrotoxin or flumazenil at various concentrations (10 nM-10 μM). Cells were then subjected to Boyden chamber assays (see, e.g., Ostrander et al, Cancer Res May 1, 2007 67; 4199, which is incorporated herein by reference). The data show that GABRA3 inhibitors picrotoxin and flumazenil suppress cell migration and invasion (FIG. 12).

### Example 7: Discussion

Our analysis of the TCGA breast cancer data uncovered a number of genes that are associated with survival. Reduced patient survival involves two major factors: resistance to therapy and metastasis. Therefore, we were careful to focus our study on genes associated with survival in our bioinformatics study that also showed an impact on metastasis when silenced and selected GABRA3 for further analysis. The GABRA3-regulated cellular pathway we identified functions in both therapy resistance and metastasis. Among the genes we found that are associated with survival, most are currently not druggable. In contrast, GABRA3 is a cell surface receptor and already has a few regulators approved by FDA and used in the clinics. The cellular pathways regulated by GABRA3 are not well-studied, thus making GABRA3 ideal for both understanding the molecular mechanisms and for translational value.

Our study opens up a number of questions regarding the role of GABA and its receptors in breast cancer metastasis. Whether GABA is required for breast cancer cells to proliferate in metastatic sites, particular in brain, has not been elucidated. We have shown that GABRA3 activates AKT pathway which is critical to both metastasis and therapy resistance. How AKT pathway is regulated by GABRA3 has not been studied. Further characterization of GABA receptors and their signaling pathways will enhance our understanding of the functions of ion channels in cancer development.

Modulations of GABA_{A} receptors are associated with sedation, ataxia, amnesia, anxiolytic and sleep activity (34, 35). There are a few GABA_{A} receptor modulators currently used in clinic. Flumazenil is a FDA-approved small molecule negative modulator of GABA_{A} receptors, which targets GABRA1, 2, 3 and 5 (36). It is used to treat idiopathic hypersomnia and improve vigilance. It is worthwhile to determine whether flumazenil and other chloride channel blockers can be used as therapeutics to treat breast cancer metastasis.

We also found that GABRA3 message undergoes A-to-I RNA editing and that the RNA-edited GABRA3 can suppress cell invasion. To our knowledge, this is the first time RNA editing has been shown to play critical roles in breast cancer progression and metastasis. RNA editing as a therapeutic target in cancer has not been studied. Interferons upregulate ADAR1 expression which we have shown is responsible for GABRA3 editing in breast cancer cells (37-39). Recombinant interferon-β (IFN-β) has been shown in clinical trials to improve clinical benefits and overall survival in metastatic breast cancer patients with minimal residual disease after chemotherapy or with disseminate disease non-progressing during endocrine therapy (40-42). Thus, the combination of targeting GABRA3 function and upregulating A-to-I editing of GABRA3 mRNAs may further improve therapeutic effects.

Each and every patent, patent application, and publication, including publications listed below, and publically available peptide sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

Sequence Listing Free Text

| SEQ ID NO | FREE TEXT |
|---|---|
| 1 | <213> Artificial Sequence |
| | <223> primer |
| 2 | <213> Artificial Sequence |
| | <223> primer |

### REFERENCES

1. Duran C, Thompson C H, Xiao Q, Hartzell H C. Chloride channels: often enigmatic, rarely predictable. Annu. Rev. Physiol. 2010; 72:95-121.
2. Watanabe M, Maemura K, Oki K, Shiraishi N, Shibayama Y, Katsu K. Gamma-aminobutyric acid (GABA) and cell proliferation: focus on cancer cells. Histol. Histopathol. 2006; 21:1135-1141.
3. Vithlani M, Terunuma M, Moss S J. The dynamic modulation of GABA_{A} receptor trafficking and its role in regulating the plasticity of inhibitory synapses. Physiol. Rev. 2011; 91:1009-1022.
4. Neman J, Termini J, Wilcznski S, Vaidehi N, Choy C, Kowolik C M, Li H, Hambrecht A C, Roberts E, Jandial R. Human breast cancer metastases to the brain display GABAergic properties in the neural niche. Proc. Natl. Acad. Sci. U.S.A. 2014; 111:984-989.
5. Bass B L, Weintraub H. A developmentally regulated activity that unwinds RNA duplexes. Cell. 1987; 48:607-13.
6. Rebagliati M R, Melton D A. Antisense RNA injections in fertilized frog eggs reveal an RNA duplex unwinding activity. Cell. 1987; 48:599-605.
7. Bass B L, Weintraub H. An unwinding activity that covalently modifies its double-stranded RNA substrate. Cell. 1988; 55:1089-98.
8. Wagner R W, Smith J E, Cooperman B S, Nishikura K. A double-stranded RNA unwinding activity introduces structural alterations by means of adenosine to inosine conversions in mammalian cells and Xenopus eggs. Proc Natl Acad Sci USA. 1989; 86:2647-51.
9. Nishikura K. Editor meets silencer: crosstalk between RNA editing and RNA interference. Nat Rev Mol Cell Biol. 2006; 7:919-31.
10. Nishikura K. Functions and regulation of RNA editing by ADAR deaminases. Annu Rev Biochem. 2010; 79:321-49.
11. Hood J L, Emeson R B. Editing of neurotransmitter receptor and ion channel RNA in the nervous system. Curr. Top. Microbiol. Immunol. 2012; 353:61-90.
12. Kim U, Wang Y, Sanford T, Zeng Y, Nishikura K. Molecular cloning of cDNA for double-stranded RNA adenosine deaminase, a candidate enzyme for nuclear RNA editing. Proc Natl Acad Sci USA. 1994; 91:11457-61.
13. Gerber A, O'Connell M A, Keller W. Two forms of human double-stranded RNA-specific editase 1 (hRED1) generated by the insertion of an Alu cassette. RNA. 1997; 3:453-63.
14. Lai F, Chen C X, Carter K C, Nishikura K. Editing of glutamate receptor B subunit ion channel RNAs by four alternatively spliced DRADA2 double-stranded RNA adenosine deaminases. Mol Cell Biol. 1997; 17:2413-24.
15. Melcher T, Maas S, Herb A, Sprengel R, Seeburg P H, Higuchi M. A mammalian RNA editing enzyme. Nature. 1996; 379:460-4.
16. Chen C X, Cho D S, Wang Q, Lai F, Carter K C, Nishikura K. A third member of the RNA-specific adenosine deaminase gene family, ADAR3, contains both single- and double-stranded RNA binding domains. RNA. 2000; 6:755-67.
17. Melcher T, Maas S, Herb A, Sprengel R, Higuchi M, Seeburg P H. RED2, a brain-specific member of the RNA-specific adenosine deaminase family. J Biol Chem. 1996; 271:31795-8.
18. Daniel C, Wahlstedt H, Ohlson J, Bjork P, Ohman M. Adenosine-to-inosine RNA editing affects trafficking of the γ-aminobutyric acid type A (GABA_{A}) receptor. J Biol Chem. 2011; 286:2031-2040.
19. Gumireddy K, Li A, Kossenkov A V, Cai K W, Liu Q, Yan J, Xu H, Showe L, Zhang L, Huang Q. ID1 promotes breast cancer metastasis by S100A9 regulation. Molecular Cancer Research 2014; 12(9):1334-1343.
20. Gumireddy K, Li A, Yan J, Setoyama T, Johannes G J, Ørom U A, Tchou J, Liu Q, Zhang L, Speicher D W, Calin G A, Huang Q. Identification of a long noncoding RNA-associated RNP complex regulating metastasis at the translational Step. EMBO Journal 2013; 32:2672-2684.
21. Gumireddy K, Li A, Gimotty P A, Klein-Szanto A J, Showe L C, Katsaros D, Coukos G, Zhang L, Huang Q. KLF17 is a negative regulator of epithelial-mesenchymal transition and metastasis in breast cancer. Nature Cell Biology 2009; 11(11):1297-1304.
22. Huang Q, Gumireddy K, Schrier M, le Sage C, Nagel R, Nair S, Egan D A, Li A, Huang G, Klein-Szanto A J, Gimotty P A, Katsaros D, Coukos G, Zhang L, Puré E, Agami R. The microRNAs miR-373 and miR-520c Promote Tumor Invasion and Metastasis. Nature Cell Biology 2008; 10(2):202-210.
23. Heidenreich B, Rachakonda P S, Hemminki K, Kumar R. TERT promoter mutations in cancer development. Curr Opin Genet Dev 2014; 24:30-37.
24. Sun Y, Shen S, Liu X, Tang H, Wang Z, Yu Z, Li X, Wu M. MiR-429 inhibits cell growth and invasion and regulates EMT-related marker genes by targeting Onecut 2 in colorectal carcinoma. Mol Cell Biochem 2014; 90:19-30.
25. Dunne J, Mannari D, Farzaneh T, Gessner A, van Delft F W, Heidenreich O, Young B D, Gascoyne D M. AML1/ETO and POU4F1 synergy drives B-lymphoid gene expression typical of t(8;21) acute myeloid leukemia. Leukemia 2012; 26:1131-1135.
26. Torisu Y, Watanabe A, Nonaka A, Midorikawa Y, Makuuchi M, Shimamura T, Sugimura H, Niida A, Akiyama T, Iwanari H, Kodama T, Zeniya M, Aburatani H. Human homologu of NOTUM, overexpression in hepatocellular carcinoma, is regulated transcriptionally by beta-catenin/TCF. Cancer Sci 2008; 99:1139-1146.
27. McCubrey J A, Abrams S L, Fitzgerald T L, Cocco L, Martelli A M, Montalto G, Cervello M, Scalisi A, Candido S, Libra M, Steelman L S. Roles of signaling pathways in drug resistance, cancer initiating cells and cancer progression and metastasis. Adv Bio Regul 2015; 57:75-101.
28. Martini M, De Santis M C, Braccini L, Gulluni F, Hirsch E. PI3K/AKT signaling pathway and cancer: an updated review. Ann Med 2014; 46:372-382.
29. Davis N M, Sokolosky M, Stadelman K, Abrams S L, Libra M, Candido S, Nicoletti F, Polesel J, Maestro R, D'Assoro A, Drobot L, RAkus D, Gizak A, Laidler P, Dulinska-Litewka J, Basecke J, Mijatovic S, Maksimovic-Ivanic D, Montalto G, Cervello M, Fitzgerald T L, Demidenko Z, Martelli A M, Cocco L, Steelman L S, McCubrey J A. Deregulation of the EGFR/PI3K/PTEN/Akt/mTORC1 pathway in breast cancer: possibilities for therapeutic intervention. Oncotarget 2014; 2:4603-4650.
30. Stepulak A, Luksch H, Gebhardt C, Uckermann O, Marzahn J, Sifringer M, Rzeski W, Staufner C, Brocke K S, Turski L, Ikonomidou C. Expression of glutamate receptor subunits in human cancer. Histochem Cell Biol. 2009; 132:435-445.
31. Wangari-Talbot J, Wall B A, Goydos J S, Chen S. Functional effects of GRM1 suppression in human melanoma cells. Mol. Cancer Res. 2012; 10:1440-1450.
32. Lee H J, Wall B A, Wangari-Talbot J, Shin S S, Rosenberg S A, Chan J L, Namkoong J, Goydos J S, Chen S. Glutamatergic pathway targeting in melanoma; single agent and combinatorial therapies. Clin. Cancer Res. 2011; 17:7080-7092.
33. Yip D, Le M N, Chan J L, Lee J H, Mehnert J A, Yudd A, Kempf J, Shih W J, Chen S, Goydos J S. A phase 0 trial of riluzole in patients with resectable stage III and IV melanoma. Clin. Cancer Res. 2009; 15:3896-3902.
34. Crestani F, Rudolph U. Behavioral functions of GABAA receptor subtypes—the Zurich experience. Adv Pharmacol 2015; 72:37-51.
35. Fritschy J M, Panzanelli P. GABAA receptors and plasticity of inhibitory neurotransmission in the central nervous system. Eur J Neurosci 2014; 39:1845-65.
36. Mohler H. The legacy of the benzodiazepine receptor: from flumazenil to enhancing cognition in Down syndrome and social interaction in autism. Adv Pharmacol 2015; 72:1-36.
37. Yang W, Wang, Q, Kanes S J, Murray J M, Nishikura K. Altered RNA editing of serotonin 5-HT2c receptor induced by interferon: implications for depression associated with cytokine therapy. Mol. Brian Res. 2004; 124:70-78.
38. Patterson J B, Samuel C E. Expression and regulation by interferon of a double-stranded RNA specific adenosine deaminase from Human cells: evidence for two forms of the deaminase. Mol. Cell. Biol. 1995; 15:5376-5388.
39. George C X, Wagner M V, Samuel C E. Expression of interferon-inducible RNA adenosine deaminase ADAR1 during pathogen infection and mouse embryo development involves tissue-selective promoter utilization and alternative splicing. J. Biol. Chem. 2005; 280:15020-15028.
40. Carpi A, Nicolini A, Antonelli A, Ferrari P, Rossi G. Cytokines in the management of high risk or advanced breast cancer: an update and expectation. Curr. Cancer Drug Targets 2009; 9:888-903.
41. Recchia F, Sica G, Candeloro G, Necozione S, Bisegna R, Bratta M, Rea S. Beta-interferon, retinoids and tamoxifen in metastatic breast cancer: long-term follow-up of a phase II study. Oncology Report 2009; 21:1011-1016.
42. Nicolini A, Carpi A. Beta-interferon and interleukin-2 prolong more than three times the survival of 26 consecutive endocrine dependent breast cancer patients with distant metastases: an exploratory trial. Biomed. Pharmacotherapy 2005; 59:253-263.

What is claimed is:

1. A method of diagnosing and treating breast cancer in a subject comprising,
a. measuring the level of GABRA3 in a biological sample from the subject, wherein the presence of GABRA3, or an increase in the level, in the sample as compared to a control is indicative of breast cancer; and
b. treating the breast cancer by decreasing the GABRA3 levels in the subject

2. The method of claim 1, wherein the sample comprises breast tissue cells.

3. The method of claim 2, wherein the sample is a tissue biopsy.

4. The method of claim 1, wherein the control is a sample derived from normal breast tissue or cells.

5. The method of claim 1, comprising administering a GABAA receptor antagonist.

6. The method of claim 5, wherein the GABAA receptor antagonist is flumazenil, thiocolchicoside, pentetrazol, picrotoxin or topiramate.

7. The method of claim 1, comprising increasing the expression of A-to-I edited GABRA3.

8. A method of detecting the risk of breast cancer metastasis in a subject comprising, measuring the level of GABRA3 in a biological sample from the subject, wherein an increase in the level of GABRA3 in the sample as compared to a control, is indicative of an increased risk of metastasis.

9. The method of claim 8, wherein the control is a sample derived from normal breast tissue or cells.

10. The method according to claim 6, wherein the measuring step further comprises measuring the level of A-to-I RNA edited GABRA3 in a biological sample from the subject, wherein an decrease in the level of A-to-I RNA edited GABRA3 in the sample as compared to a control, is indicative of an increased risk of metastasis.

11. The method of claim 10, wherein the control is a sample derived from a patient having metastatic breast cancer.

12. A method of treating breast cancer, the method comprising inhibiting the action of GABA.

13. The method of claim 12, comprising decreasing GABRA3 levels in a subject.

14. The method of claim 12, comprising administering a GABAA receptor antagonist.

15. The method of claim 14, wherein the GABAA receptor antagonist is flumazenil, thiocolchicoside, pentetrazol, picrotoxin or topiramate.

16. The method of claim 12, comprising increasing the expression of A-to-I edited GABRA3.
